(19) United States
(12) Patent Application Publication
(10) Pub. No.: US 2024/0108230 A1
(43) Pub. Date: Apr. 4, 2024

(54) METHOD FOR EVALUATING HEALTH CONDITION INDICATOR OF SUBJECT, HOST, AND COMPUTER READABLE STORAGE MEDIUM

(71) Applicant: BOMDIC INC., New Taipei City (TW)

(72) Inventors: Yao Shiao, New Taipei City (TW); Shao Wen Tou, New Taipei City (TW); Yu-Ting Liu, New Taipei City (TW); Amy Pei-Ling Chiu, New Taipei City (TW)

(73) Assignee: BOMDIC INC., New Taipei City (TW)

(21) Appl. No.: 17/956,848

(22) Filed: Sep. 30, 2022

(51) Int. Cl. A61B 5/0205; A61B 5/00

(52) U.S. Cl. CPC A61B 5/0205; A61B 5/6801

(57) ABSTRACT

The embodiments of the disclosure provide a method for evaluating a health condition indicator of a subject, a host, and a computer readable storage medium. The method includes: obtaining a reference heart rate of the subject and determining a heart rate indicator of the subject according to the reference heart rate; determining a fitness indicator of the subject based on physiological information of the subject; determining an age indicator of the subject based on an age of the subject; and determining the health condition indicator of the subject at least based on the heart rate indicator, the fitness indicator, and the age indicator.

### BACKGROUND

### 1. Field of the Invention

The present disclosure generally relates to a health evaluation mechanism, in particular, to a method for evaluating a health condition indicator of a subject, a host, and a computer readable storage medium.

### 2. Description of Related Art

Nowadays, it is common for people to have personal insurance. When an individual applies for insurance to the insurance company, the insurance company usually needs to assess the health condition of this individual and accordingly determine whether to provide insurance service or provide which type or degree of insurance service to this individual. For example, the insurance company may determine whether to provide insurance service or provide which type or degree of insurance service to an individual based on his/her appearance (e.g., body shape and/or handicap), medical history, chronic disease, family medical history, body examination result, medical records, diagnostics, habits, leisure activities, insurance status, etc.

Based on the above information, an individual can be categorized into one of four types, e.g., standard, substandard, declinature, and preferred.

Generally speaking, for an individual categorized into the type of standard, it represents this individual has good health and moderate physique. To insurance companies, the expected mortality rate of such an individual is about the average, which means that this is an ordinary individual, neither too healthy nor too unhealthy.

For an individual categorized into the type of substandard, it represents this individual has poor physical condition, for example being overweight, or has a mild past medical history. Therefore, the insurance company estimates that the expected risk of death is higher. Although this type of individual can still apply for insurance, the insurance fee may be estimated to be higher than an individual categorized into the type of standard.

For an individual categorized into the type of declinature, it represents this individual is declined by the insurance companies due to, for example, medical history, chronic diseases, having a career with high risk.

For an individual categorized into the type of preferred, it represents that the insurance companies believe that this individual has a lower-than-average expected chance of death. Some insurance companies might give a discounted insurance fee for clients categorized into the type of preferred.

Therefore, it is crucial to design a mechanism to properly categorize people into the above types for the insurance companies to determine whether to provide insurance services to people and/or the corresponding insurance fees.

### SUMMARY OF THE INVENTION

Accordingly, the disclosure is directed to a method for evaluating a health condition indicator of a subject, a host, and a computer readable storage medium, which may be used to solve the above technical problems.

The embodiments of the disclosure provide a method for evaluating a health condition indicator of a subject, adapted to a host, including: obtaining a reference heart rate of the subject and determining a heart rate indicator of the subject according to the reference heart rate; determining a fitness indicator of the subject based on physiological information of the subject; determining an age indicator of the subject based on an age of the subject; and determining the health condition indicator of the subject at least based on the heart rate indicator, the fitness indicator, and the age indicator.

The embodiments of the disclosure provide a host including a storage circuit and a processor. The storage circuit stores a program code. The processor is coupled to the storage circuit and accesses the program code to perform: obtaining a reference heart rate of the subject and determining a heart rate indicator of the subject according to the reference heart rate; determining a fitness indicator of the subject based on physiological information of the subject; determining an age indicator of the subject based on an age of the subject; and determining the health condition indicator of the subject at least based on the heart rate indicator, the fitness indicator, and the age indicator.

The embodiments of the disclosure provide a computer readable storage medium, the computer readable storage medium recording an executable computer program, the executable computer program being loaded by a host to perform steps of: obtaining a reference heart rate of the subject and determining a heart rate indicator of the subject according to the reference heart rate; determining a fitness indicator of the subject based on physiological information of the subject; determining an age indicator of the subject based on an age of the subject; and determining a health condition indicator of the subject at least based on the heart rate indicator, the fitness indicator, and the age indicator.

### BRIEF DESCRIPTION OF THE DRAWINGS

The accompanying drawings are included to provide a further understanding of the invention and are incorporated in and constitute a part of this specification. The drawings illustrate embodiments of the invention and, together with the description, serve to explain the principles of the disclosure.

FIG. 1 shows a schematic diagram of a host according to an embodiment of the disclosure.

FIG. 2 shows a flow chart of the method for evaluating a health condition indicator of a subject according to an embodiment of the disclosure.

FIG. 3 shows a schematic diagram of deriving the heart rate indicator model according to an embodiment of the disclosure.

FIG. 4 shows a schematic diagram of determining the fitness indicator according to an embodiment of the disclosure.

FIG. 5 shows a schematic diagram of determining the physical activity indicator according to an embodiment of the disclosure.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

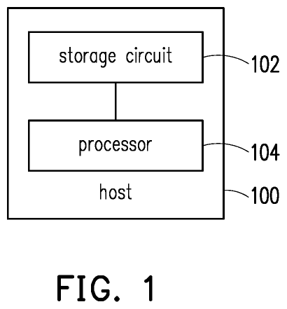

See FIG. 1, which shows a schematic diagram of a host according to an embodiment of the disclosure. In various embodiments, the host 100 can be implemented as any smart device and/or computer devices, but the disclosure is not limited thereto.

In FIG. 1, the host 100 includes a storage circuit 102 and a processor 104. The storage circuit 102 is one or a combination of a stationary or mobile random access memory (RAM), read-only memory (ROM), flash memory, hard disk, or any other similar device, and which records a plurality of modules and/or program codes that can be executed by the processor 104.

The processor 104 may be coupled with the storage circuit 102, and the processor 104 may be, for example, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGAs) circuits, any other type of integrated circuit (IC), a state machine, and the like.

In the embodiments of the disclosure, the processor 104 may access the modules and/or program codes stored in the storage circuit 102 to implement the method for evaluating a health condition indicator of a subject provided in the disclosure, which would be further discussed in the following.

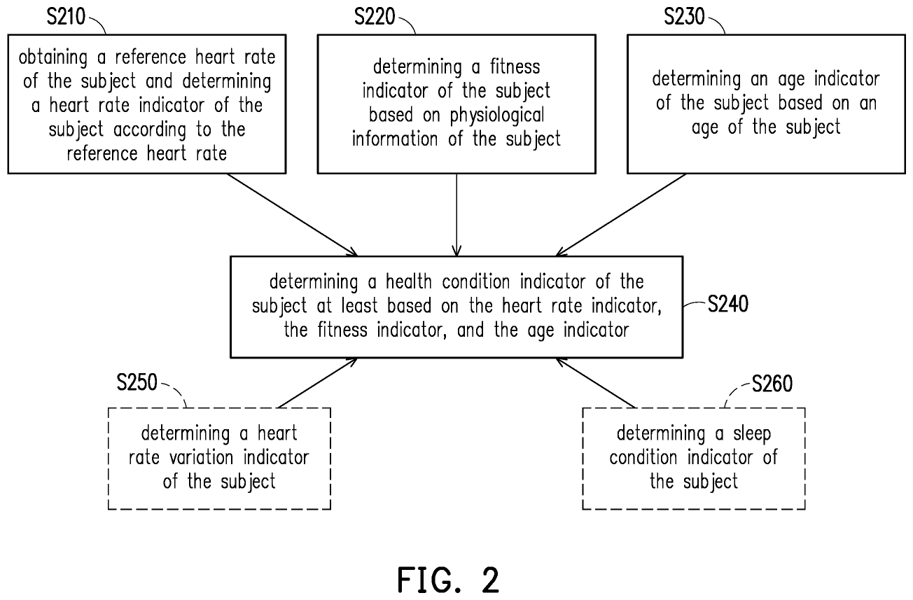

See FIG. 2, which shows a flow chart of the method for evaluating a health condition indicator of a subject according to an embodiment of the disclosure. The method of this embodiment may be executed by the host 100 in FIG. 1, and the details of each step in FIG. 2 will be described below with the components shown in FIG. 1.

In step S210, the processor 104 obtains a reference heart rate of the subject and determines a heart rate indicator (referred to as GM_rest) of the subject according to the reference heart rate.

In the embodiments of the disclosure, the subject can be a person assessed by the insurance companies, or other person whose health condition is being assessed, but the disclosure is not limited thereto.

In one embodiment, the reference heart rate may be a resting heart rate (referred to as HR resting) of the subject. In one embodiment, the resting heart rate may be the heart rate measured at the time when the subject wakes up, asleep, and/or in a supine position or a steady state, but the disclosure is not limited thereto. In the embodiments of the disclosure, the resting heart rate of the subject can be measured by any existing technology used for measuring the resting heart rate of a person. Details associated with the resting heart rate can be referred to “Aune, D et al. “Resting heart rate and the risk of cardiovascular disease, total cancer, and all-cause mortality—A systematic review and dose-response meta-analysis of prospective studies.”

In one embodiment, during determining the heart rate indicator of the subject according to the reference heart rate, the processor 104 can perform: converting the reference heart rate into a heart rate factor (referred to as REST), wherein the heart rate factor ranges between 0 and 1; and inputting the heart rate factor into a heart rate indicator model, wherein the heart rate indicator model outputs the heart rate indicator in response to the heart rate factor.

In one embodiment, the processor 104 may firstly determine a heart rate upper limit (referred to as HR_UL) and a heart rate lower limit (referred to as HR LL). In one embodiment, the heart rate upper limit can be predetermined to be a value corresponding to a high resting heart rate, for example 100. Similarly, the heart rate lower limit can be predetermined to be a value corresponding to a low resting heart rate, for example 40.

In another embodiment, the heart rate upper limit and lower limit can also be determined in a statistical way. For example, the processor 104 may obtain a group of people and their individual heart rate and respectively use the maximum and minimum values of these heart rates as the heart rate upper limit and the heart rate lower limit, but the disclosure is not limited thereto. In still another embodiment, the values of the heart rate upper and lower limit may be automatically updated according to a cloud heart rate database.

In one embodiment, the heart rate factor (i.e., REST) can be determined to be a value positively related to the resting heart rate. That is, the higher the resting heart rate, the higher the heart rate factor, and vice versa.

In one embodiment, the heart rate factor can be determined based on the following:

$$REST = \frac{HR\_rest - HR\_LL}{HR\_UL - HR\_LL}$$

but the disclosure is not limited thereto.

In one embodiment, the processor 104 may obtain a mortality and a reference resting heart rate of each of a plurality of reference subjects and accordingly derive the heart rate indicator model.

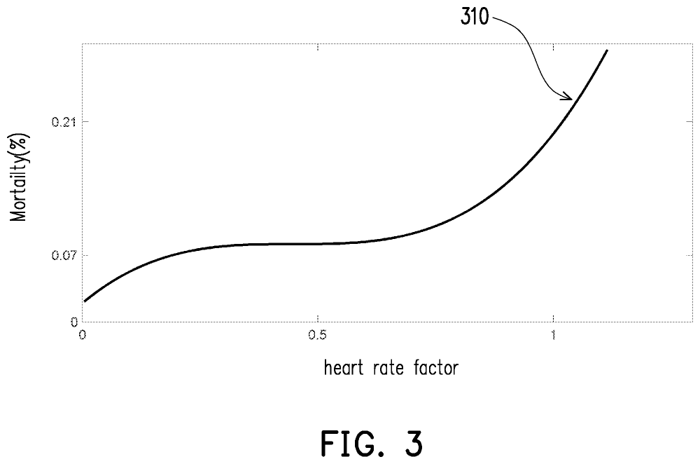

See FIG. 3, which shows a schematic diagram of deriving the heart rate indicator model according to an embodiment of the disclosure. In FIG. 3, the processor 104 may obtain the mortality and the reference resting heart rate of each reference subject from the related statistical health data, and the processor 104 may convert the reference resting heart rate of each reference subject into the corresponding heart rate factor. Next, based on the mortality and the heart rate factor of each reference subject, the processor 104 can perform operations such as line fitting to obtain the heart rate indicator model 310 exemplarily shown in FIG. 3. In this case, once the heart rate indicator of a person is obtained, the processor 104 may accordingly determine the corresponding mortality based on the heart rate indicator model 310.

In one embodiment, the heart rate indicator model 310 can be characterized by a function as follows:

GM_rest=(0.003(REST−12)3+5),

but the disclosure is not limited thereto.

In this case, the processor 104 may input the obtained heart rate factor of the subject into the heart rate indicator model 310, and the heart rate indicator model 310 can output the corresponding heart rate indicator (i.e., GM_rest) in response to the heart rate factor, but the disclosure is not limited thereto.

In step S220, the processor 104 determines a fitness indicator (referred to as GM_fitness) of the subject based on physiological information of the subject.

In one embodiment, the processor 104 determines a physical activity indicator of the subject according to the physiological information of the subject and converts the physical activity indicator into a hazard ratio as the fitness indicator of the subject, wherein the hazard ratio ranges between 0 and 1.

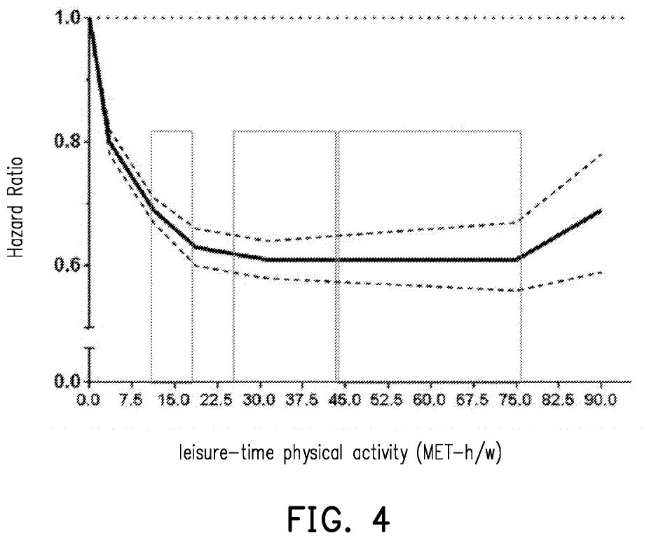

See FIG. 4, which shows a schematic diagram of determining the fitness indicator according to an embodiment of the disclosure. In FIG. 4, the content therein is reproduced based on the document of “Kraus W E, Powell K E, Haskell W L, et al. Physical Activity, All-Cause and Cardiovascular Mortality, and Cardiovascular Disease”. In the embodiment, the considered physical activity indicator may be the metabolic equivalent of task (MET) of the subject based on the related physiological information of the subject, wherein the MET of the subject can be obtained by any related existing technology.

In FIG. 4, the MET can be the leisure-time physical activity hours per week. As can be seen from FIG. 4, in the region where MET is lower than 75 hours per week, as the leisure-time physical activity hours per week of a person increases, the hazard ratio of this person gets lower. However, when the leisure-time physical activity hours per week of a person is more than 75 hours per week, the hazard ratio of this person gets higher.

In one embodiment, with the obtained physical activity indicator (e.g., MET), the processor 104 may convert the obtained physical activity indicator into the corresponding hazard ratio based on FIG. 4. For example, if the MET of the subject is 45, the processor 104 may determine the corresponding hazard ratio (e.g., 0.6) as the fitness indicator of the subject, but the disclosure is not limited thereto.

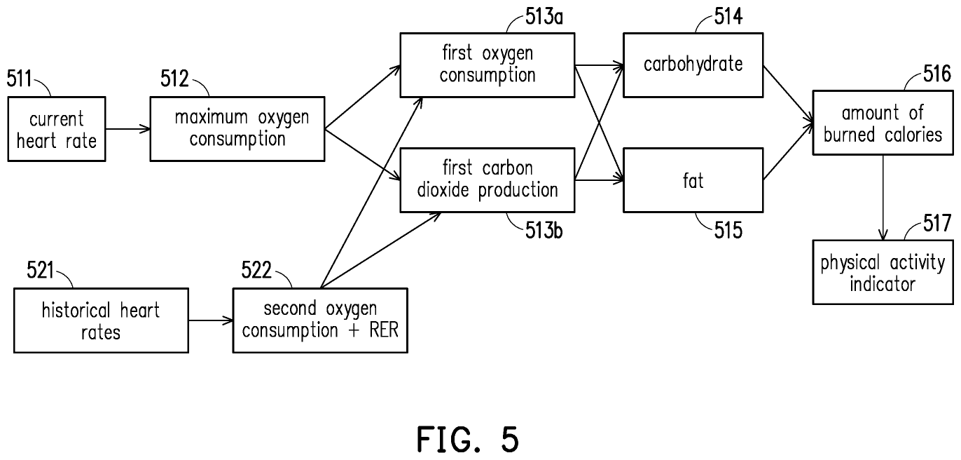

In other embodiments, the physical activity indicator can be obtained in other ways. See FIG. 5, which shows a schematic diagram of determining the physical activity indicator according to an embodiment of the disclosure.

In the scenario of FIG. 5, the considered physiological information of the subject comprises a current heart rate 511 of the subject, a plurality of historical heart rates 521 of the subject. In FIG. 5, the processor 104 can determine a maximum oxygen consumption 512 based on the current heart rate 511 of the subject and accordingly determine a first oxygen consumption 513a and a first carbon dioxide production 513b. In the embodiment, how the maximum oxygen consumption 512 is determined based on the current heart rate 511 and how to derive the first oxygen consumption 513a and the first carbon dioxide production 513b based on the maximum oxygen consumption 512 can be referred to the related existing technology.

In some embodiments, although the current heart rate 511 can be used to derive the first oxygen consumption 513a and the first carbon dioxide production 513b, it may be inaccurate to directly use the first oxygen consumption 513a and the first carbon dioxide production 513b to derive the amount of burned calories 516 corresponding to the current heart rate 511. For example, when the subject takes a rest after experiencing an intense workout, the current heart rate may be still high, but the oxygen consumption and the carbon dioxide production may not be as high as the first oxygen consumption 513a and the first carbon dioxide production 513b.

Therefore, the processor 104 may further consider the historical heart rates 521 to correct the first oxygen consumption 513a and the first carbon dioxide production 513b. In one embodiment, the processor 104 can determine a second oxygen consumption and a respiratory exchange ratio (RER) based on the historical heart rates 521 of the subject and accordingly determine a second carbon dioxide production. In the embodiments, the historical heart rates 521 may be the heart rates of the subject in the past few seconds (e.g., seconds), but the disclosure is not limited thereto.

In one embodiment, in response to determining that the historical heart rates 521 indicate that the heart rate of the subject is decreasing, it represents that the subject may be resting. In this case, the actual oxygen consumption should be lower than the first oxygen consumption 513a, and the actual carbon dioxide production should be lower than the first carbon dioxide production 513b. In this case, the processor 104 may determine the second oxygen consumption via decreasing the first oxygen consumption 513a by a first amount, wherein the second oxygen consumption can be understood as the oxygen consumption estimated to be closer to the actual oxygen consumption.

In one embodiment, the first carbon dioxide production 513b can be derived via multiplying the first oxygen consumption 513a with a predetermined RER. Since the actual carbon dioxide production should be lower than the first carbon dioxide production 513b, the processor 104 can further determine a (new) RER via decreasing the predetermined RER by a second amount. Next, the processor 104 can multiply the second oxygen consumption with the (new) RER to obtain the second carbon dioxide production, but the disclosure is not limited thereto.

Afterward, the processor 104 can determine a carbohydrate usage 514 and a fat usage 515 based on the second oxygen consumption and the second carbon dioxide production and accordingly determine an amount of burned calories 516. In the embodiments of the disclosure, how to determine the carbohydrate usage 514 and the fat usage 515 based on the second oxygen consumption and the second carbon dioxide production and how to accordingly determine the amount of burned calories 516 can be referred to the related existing technology.

In one embodiment, the processor 104 can determine the physical activity indicator of the subject based on the amount of burned calories 516 (referred to as Kcal). For example, the physical activity indicator of the subject can be the MET characterized as follows:

MET=Kcal*200/0.8/BW,

wherein BW is the body weight of the subject, but the disclosure is not limited thereto.

Accordingly, the physical activity indicator reflecting the activity condition of the subject more properly can be determined.

In step S230, the processor 104 determines an age indicator (referred to as GM_age) of the subject based on the age of the subject.

In one embodiment, the processor 104 may determine a deviation degree of the age (referred to as AGE) of the subject relative to an average age (referred to as AGE_avg) as the age indicator of the subject, wherein the deviation degree ranges between 0 and 1.

In one embodiment, the deviation degree may be a positive value negatively related to a difference between AGE and AGE_avg if AGE is lower than AGE_avg. In another embodiment, the deviation degree may be a negative value positively related to a difference between AGE and AGE_avg if AGE is higher than AGE_avg.

In one embodiment, the deviation degree may be characterized as

$$\frac{AGE\_avg - AGE}{AGE\_avg} ,$$

but the disclosure is not limited thereto.

In one embodiment, AGE_avg may be the average age of the group to which the subject belongs, for example the average age of the people in the country/region of the subject, but the disclosure is not limited thereto. In another embodiment, AGE_avg may be automatically updated according to a cloud AGE_avg database.

In step S240, the processor 104 determines a health condition indicator (referred to as GM_score) of the subject at least based on the heart rate indicator, the fitness indicator, and the age indicator.

In one embodiment, the processor 104 can linearly combine the heart rate indicator, the fitness indicator, and the age indicator into the health condition indicator of the subject. For example, the health condition indicator of the subject can be characterized as:

GM_score=α*GM_rest+β*GM_fitness+γ*GM_age

wherein α, β, γ can be the coefficients respectively corresponding to GM_rest, GM_fitness, and GM_age.

In different embodiments, α, β, γ can be determined in different ways. In one embodiment, the designer can determine α, β, γ based on the requirements thereof. For example, if the designer believes that GM_rest and GM_fitness are more important for evaluating the health condition of the subject, the designer may determine the corresponding α, β to be higher than γ. In one embodiment, α, β, γ may be, for example, 1.5, 1.5, 1, respectively.

In another embodiment, if the data that specifies the relation between the mortality and/or risk of death between each of the resting heart rate, physical activity, and age are available, the processor 104 may accordingly perform operations such as linear regression to determine α, β, γ, but the disclosure is not limited thereto.

In some embodiments, α, β, γ can be determined to limit GM_score to be between an upper limit (e.g., 5) and a lower limit (e.g., 1), but the disclosure is not limited thereto.

In some embodiments, before performing step S240, the processor 104 may additionally perform steps S250 and S260.

In step S250, the processor 104 determines a heart rate variation indicator (referred to as GM_HRV) of the subject. In one embodiment, the processor 104 obtains a heart rate variation detected by a wearable device worn on the subject and converts the heart rate variation into the heart rate variation indicator of the subject, wherein the heart rate variation indicator of the subject ranges between 0 and 1.

In one embodiment, the wearable device may be, for example, a smart bracelet/watch worn by the subject for several hours/days for determining the heart rate variation of the subject, but the disclosure is not limited thereto.

In the embodiments of the disclosure, the heart rate variation indicator may be positively related to the heart rate variation of the subject. That is, the higher the heart rate variation of the subject, the higher the heart rate variation indicator, and vice versa.

In step S260, the processor 104 determines a sleep condition indicator (referred to as GM_sleep) of the subject.

In one embodiment, the processor 104 may perform: obtaining a circadian status (referred to as GM_circadian) of the subject; obtaining an obstructive sleep apnoea (OSA) status (referred to as GM_OSA) of the subject; obtaining a sleep stage status (referred to as GM_sleepstage) of the subject. In the embodiments of the disclosure, the circadian status, the OSA status, and the sleep stage status can be obtained by the wearable device worn on the subject for several hours/days.

For example, the wearable device can determine which of the hours in a day correspond to the duration where the subject is in activity and which of the hours in a day correspond to the duration where the subject is sleeping.

In one embodiment, the OSA status of the subject can be detected by analysing which part of the sleeping time of the subject experiencing an OSA.

In one embodiment, the rapid eye movement (REM) stage and the non-REM stage of the subject can be detected by analysing the heart rate and the accelerations detected during the sleeping time of the subject, and hence the sleep stage status can be obtained. In some embodiment, since there are some existing prior arts can determine a sleeping score of a person, the processor 104 may simply convert the sleeping score into the corresponding sleep stage status, but the disclosure is not limited thereto.

In one embodiment, the processor 104 determines the sleep condition indicator of the subject based on at least one of the circadian statuses of the subject, the obstructive sleep apnoea status of the subject, and the sleep stage status of the subject, wherein the sleep condition indicator of the subject ranges between 0 and 1.

In one embodiment, the processor 104 may linearly combine the circadian status of the subject, the OSA status of the subject, and the sleep stage status of the subject into the sleep condition indicator.

In one embodiment, the processor 104 may determine a regular circadian and accordingly determine a deviation degree of a current circadian of the subject relative to the regular circadian as the circadian status. For example, if the current circadian indicates that the subject does not have regular sleep time, the processor 104 may determine the circadian status to be a lower value. On the other hand, if the current circadian indicates that the subject has regular sleep time, the processor 104 may determine the circadian status to be a higher value.

In one embodiment, if the processor 104 determines that the subject suffers from OSA badly, the processor 104 may determine the OSA status to be a lower value. On the other hand, if the processor 104 determines that the subject does not suffer from the OSA, the processor 104 may determine the OSA status to be a higher value.

In one embodiment, if the processor 104 determines that the sleeping stages of the subject are properly distributed within the sleep time of the subject, the processor 104 may determine the sleep stage status to be a higher value. On the other hand, if the processor 104 determines that the sleeping stages of the subject are not properly distributed within the sleep time of the subject, the processor 104 may determine the sleep stage status to be a lower value.

In one embodiment, the sleep condition indicator may be characterized as follows:

GMₛₗₑₑₚ=a*GM_{circadian}+b*GM_{sleepstage}+C*GM_OSA

wherein a, b, c are the coefficients respectively corresponding to GM_circadian, GM_OSA, and GM_sleepstage, and a, b, c can be determined based on the requirements of the designer. In one embodiment, a, b, c may be, for example, 0.3, 0.3, 0.4, respectively, but the disclosure is not limited thereto.

With GM_HR and GM_sleep, when the processor 104 performs step S240, the processor 104 may determine the health condition indicator of the subject based on the heart rate indicator, the fitness indicator, the age indicator, and at least one of the heart rate variation indicator and the sleep condition indicator.

In one embodiment the health condition indicator can be characterized as follows:

GM_{score}−α*GMᵣₑₛₜ+β*GM_{fitness}+γ*GM_{age}+δ*GM_HRV+ε*GM_sleep

wherein δ and ε are the coefficients corresponding to GM_HRV and GM_sleep, respectively, and δ and ε can be determined based on the principle similar to determining α, β, γ. In one embodiment, δ and ε may be, for example, 0.5 and 0.5, respectively.

The disclosure further provides a computer readable storage medium for executing the method for adjusting a virtual object. The computer readable storage medium is composed of a plurality of program instructions (for example, a setting program instruction and a deployment program instruction) embodied therein. These program instructions can be loaded into the host 100 and executed by the same to execute the method for adjusting a virtual object and the functions of the host 100 described above.

In summary, the embodiments of the disclosure provide a solution to properly determine the health condition indicators of individuals, where the healthier individuals may have better health condition indicators and the less healthy individuals may have worse health condition indicators. Accordingly, the insurance companies can categorize people into the above types in a novel, efficient and objective way.

It will be apparent to those skilled in the art that various modifications and variations can be made to the structure of the present invention without departing from the scope or spirit of the disclosure. In view of the foregoing, it is intended that the present disclosure cover modifications and variations of this invention provided they fall within the scope of the following claims and their equivalents.

What is claimed is:

1. A method for evaluating a health condition indicator of a subject, adapted to a host, comprising:
obtaining a reference heart rate of the subject and determining a heart rate indicator of the subject according to the reference heart rate;
determining a fitness indicator of the subject based on physiological information of the subject;
determining an age indicator of the subject based on an age of the subject; and
determining the health condition indicator of the subject at least based on the heart rate indicator, the fitness indicator, and the age indicator.

2. The method according to claim 1, wherein the reference heart rate is a resting heart rate of the subject.

3. The method according to claim 2, wherein the step of determining the heart rate indicator of the subject according to the reference heart rate comprises:
converting the reference heart rate into a heart rate factor, wherein the heart rate factor ranges between 0 and 1;
inputting the heart rate factor into a heart rate indicator model, wherein the heart rate indicator model outputs the heart rate indicator in response to the heart rate factor.

4. The method according to claim 3, further comprising:
obtaining a mortality and a reference resting heart rate of each of a plurality of reference subjects and accordingly deriving the heart rate indicator model.

5. The method according to claim 1, wherein the step of determining the fitness indicator of the subject based on the physiological information of the subject comprises:
determining a physical activity indicator of the subject according to the physiological information of the subject;
converting the physical activity indicator into a hazard ratio as the fitness indicator of the subject, wherein the hazard ratio ranges between 0 and 1.

6. The method according to claim 5, wherein the physiological information of the subject comprises a current heart rate of the subject and a plurality of historical heart rates of the subject, and the step of determining the physical activity indicator of the subject according to the physiological information of the subject comprises:
determining a maximum oxygen consumption based on the current heart rate of the subject and accordingly determining a first oxygen consumption and a first carbon dioxide production;
determining a second oxygen consumption and a respiratory exchange ratio based on the historical heart rates of the subject and accordingly determining a second carbon dioxide production;
determining a carbohydrate usage and a fat usage based on the second oxygen consumption and the second carbon dioxide production and accordingly determining an amount of burned calories; and
determining the physical activity indicator of the subject based on the amount of burned calories.

7. The method according to claim 1, wherein the step of determining the age indicator of the subject based on the age of the subject comprises:
determining a deviation degree of the age of the subject relative to an average age as the age indicator of the subject, wherein the deviation degree ranges between 0 and 1.

8. The method according to claim 1, wherein the step of determining the health condition indicator of the subject at least based on the heart rate indicator, the fitness indicator, and the age indicator comprises:
combining the heart rate indicator, the fitness indicator, and the age indicator into the health condition indicator of the subject.

9. The method according to claim 8, comprising:
linearly combining the heart rate indicator, the fitness indicator, and the age indicator into the health condition indicator of the subject.

10. The method according to claim 1, further comprising:
determining a heart rate variation indicator of the subject;
determining a sleep condition indicator of the subject, and the step of determining the health condition indicator of the subject at least based on the heart rate indicator, the fitness indicator, and the age indicator comprises:
determining the health condition indicator of the subject based on the heart rate indicator, the fitness indicator, the age indicator, and at least one of the heart rate variation indicator and the sleep condition indicator.

11. The method according to claim 10, wherein the step of determining the heart rate variation indicator of the subject comprises:
obtaining a heart rate variation detected by a wearable device worn on the subject; and
converting the heart rate variation into the heart rate variation indicator of the subject, wherein the heart rate variation indicator of the subject ranges between 0 and 1.

12. The method according to claim 10, wherein the step of determining the sleep condition indicator of the subject comprises:
obtaining a circadian status of the subject;
obtaining an obstructive sleep apnoea status of the subject;
obtaining a sleep stage status of the subject; and
determining the sleep condition indicator of the subject based on at least one of the circadian status of the subject, the obstructive sleep apnoea status of the subject, and the sleep stage status of the subject, wherein the sleep condition indicator of the subject ranges between 0 and 1.

13. A host, comprising:
a non-transitory storage circuit, storing a program code; and
a processor, coupled to the non-transitory storage circuit and accessing the program code to perform:
obtaining a reference heart rate of the subject and determining a heart rate indicator of the subject according to the reference heart rate;
determining a fitness indicator of the subject based on physiological information of the subject;
determining an age indicator of the subject based on an age of the subject; and
determining a health condition indicator of the subject at least based on the heart rate indicator, the fitness indicator, and the age indicator.

14. The host according to claim 13, wherein the reference heart rate is a resting heart rate of the subject, and the processor performs:
converting the reference heart rate into a heart rate factor, wherein the heart rate factor ranges between 0 and 1;
inputting the heart rate factor into a heart rate indicator model, wherein the heart rate indicator model outputs the heart rate indicator in response to the heart rate factor.

15. The host according to claim 13, wherein the processor performs:
determining a physical activity indicator of the subject according to the physiological information of the subject;
converting the physical activity indicator into a hazard ratio as the fitness indicator of the subject, wherein the hazard ratio ranges between 0 and 1.

16. The host according to claim 13, wherein the processor performs:
determining a deviation degree of the age of the subject relative to an average age as the age indicator of the subject, wherein the deviation degree ranges between 0 and 1.

17. The host according to claim 13, wherein the processor performs:
combining the heart rate indicator, the fitness indicator, and the age indicator into the health condition indicator of the subject.

18. The host according to claim 13, wherein the processor further performs:
determining a heart rate variation indicator of the subject;
determining a sleep condition indicator of the subject, and the step of determining the health condition indicator of the subject at least based on the heart rate indicator, the fitness indicator, and the age indicator comprises:
determining the health condition indicator of the subject based on the heart rate indicator, the fitness indicator, and the age indicator and at least one of the heart rate variation indicator and the sleep condition indicator.

19. The host according to claim 18, wherein the processor performs:
obtaining a heart rate variation detected by a wearable device worn on the subject;
converting the heart rate variation into the heart rate variation indicator of the subject, wherein the heart rate variation indicator of the subject ranges between 0 and 1;
obtaining a circadian status of the subject;
obtaining an obstructive sleep apnoea status of the subject;
obtaining a sleep stage status of the subject; and
determining the sleep condition indicator of the subject based on at least one of the circadian status of the subject, the obstructive sleep apnoea status of the subject, and the sleep stage status of the subject, wherein the sleep condition indicator of the subject ranges between 0 and 1.

20. A non-transitory computer readable storage medium, the computer readable storage medium recording an executable computer program, the executable computer program being loaded by a host to perform steps of:
obtaining a reference heart rate of the subject and determining a heart rate indicator of the subject according to the reference heart rate;
determining a fitness indicator of the subject based on physiological information of the subject;
determining an age indicator of the subject based on an age of the subject; and
determining a health condition indicator of the subject at least based on the heart rate indicator, the fitness indicator, and the age indicator.
